(19) United States
(12) Patent Application Publication
(10) Pub. No.: US 2006/0275345 A1
(43) Pub. Date: Dec. 7, 2006

(54) Collagen hydrolysate

(75) Inventors: Michael Butzengeiger, Frankenthal (DE); Bernd Eggersgluss, Hirschhorn (DE)

Correspondence Address: LEYDIG VOIT & MAYER, LTD, 700 THIRTEENTH ST. NW, SUITE 300, WASHINGTON, DC, 20005-3960, US

(73) Assignee: Gelita AG, Eberbach (DE)

(21) Appl. No.: 11/356,415

(22) Filed: Feb. 17, 2006

Related U.S. Application Data
(63) Continuation of application No. PCT/EP04/08864, filed on Aug. 6, 2004.

(30) Foreign Application Priority Data
Aug. 21, 2003 (DE) 103 39 180.0

(51) Int. Cl. A61K 38/39; A61K 47/00

(52) U.S. Cl. 424/439; 514/2

(57) ABSTRACT

The use of collagen hydrolysate as dietary supplement to baby food, particularly to improve the general state of health in infants and bring about a reduction in restlessness, vomiting after food intake and flatulence, is disclosed.

### CROSS REFERENCE TO RELATED APPLICATIONS

This application is a continuation of international application number PCT/EP2004/008864 filed on Aug. 6, 2004, that claims the benefit of German application number 103 39 180.0, filed on Aug. 21, 2003, both which are incorporated herein by reference in their entirety and for all purposes.

### BACKGROUND OF THE INVENTION

The invention relates to novel therapeutic uses of collagen hydrolysate as dietary supplement for babies.

Collagen hydrolysate is a denatured and partially hydrolyzed protein, obtained from collagenic substances of vertebrates, in particular, mammals, poultry or fish. Peptides having a different structure and also a different biological effect result from collagen hydrolysis. Collagen hydrolysate contains approximately twice as many of the amino acids lysine, glycine and glutamine as the average food protein. Collagen hydrolysate is also rich in the amino acids proline and hydroxyproline. Hydroxyproline has not been detected in any significant concentration in any other foodstuff. The above-mentioned amino acids are essential for the formation of human collagen, i.e., for the connective tissue metabolism, in particular, the bone and cartilage metabolism.

Gelatin-containing preparations have, therefore, been adopted in the treatment of degenerative joint disease and, in the same way, hydrolyzed collagen in the treatment of osteoporosis in adults (see U.S. Pat. No. 5,948,766 A).

It has also been proven that the intake of milk containing gelatin results in growth promotion and thriving in babies and infants.

In DE 32 37 077 C2, it was suggested that instant teas be produced on the basis of cold-soluble gelatin with molecular weights of from 2,000 to 10,000 as carrier substance instead of on the basis of sucrose and/or dextrose, so as to avoid, in the case of excessive consumption of these teas, particularly by babies and infants, the caries problems otherwise caused by the sugars previously used as carrier substances.

Moreover, it was described how these instant teas are suitable for use in babies' and children's nutrition.

It was emphasized there that it is particularly advantageous that a very low nutrient content is present in the instant tea owing to the use of the hydrolyzed gelatin as carrier substance, and, therefore, the body is not given any additional food in an unintentional and uncontrolled way.

The use of gelatin hydrolysate in combination with a further protein hydrolysate, in particular, whey hydrolysate, was already known from DE 24 05 589 C3. The gelatin is used there as auxiliary agent in obtaining the whey hydrolysate and in the hydrolysis reaction avoids the formation of decomposition products with an intensive bitter taste. It is also emphasized there that gelatin hydrolysate alone as food is disadvantageous as it has only a low content of essential amino acids. In order to upgrade the value of the protein product, a protein from the group consisting of whey, milk, soya, maize, potato, wheat, oil, nut kernel, sunflower seed and egg proteins is used there, in accordance with the invention, in the presence of gelatin or gelatin decomposition products. These decomposition products are then also recommended as food supplement. Food for children and dietetic foodstuffs are also mentioned there.

The gelatin is accorded only the function of avoiding bitter substances as decomposition products.

From U.S. Pat. No. 5,948,766 A it is known that tasteless, hydrolyzed collagen from gelatin, generally animal collagenic connective tissue, having an average molecular weight of from 1 to 40 kD, may be used for the preparation of agents for the treatment of osteoporosis.

### BRIEF SUMMARY OF THE INVENTION

Hydrolyzed collagen and hydrolyzed gelatin are essentially identical. In the case of hydrolyzed gelatin, only the intermediate step of obtaining and isolating the gelatin is interposed before reducing by means of further hydrolysis the polypeptide chains to molecular weights or chain lengths which correspond to those of the hydrolyzed collagen, also referred to as collagen hydrolysate. Collagen hydrolysate and gelatin hydrolysate are, therefore, often used as synonyms.

In accordance with the present invention, collagen hydrolysate produced in an enzymatic proteolytic process is particularly preferred.

Although the low nutritional value of the gelatin as such and, consequently, also of the gelatin or collagen hydrolysate has always been pointed out in the prior art, it has now, surprisingly, been found that collagen hydrolysate produces surprising therapeutic effects as dietary supplement to baby food.

A study conducted by the inventors proved that the addition of collagen hydrolysate to the baby food results in an elevated, better general state of health in babies and, quite specifically, in a lessening of the restlessness of the babies. Furthermore, it has, surprisingly, been found that the addition of collagen hydrolysate to the baby food causes flatulence to be reduced in the babies. It has also been found that vomiting in babies after food intake has significantly decreased when collagen hydrolysate is added to the baby food.

Owing to use of collagen hydrolysate in accordance with the invention, mineralization of the skeleton was accelerated in babies.

Further astonishing results were observed in the maturing of the hips in babies, which is accelerated. In particular, quicker bone formation in the femoral head was observed.

The above results are all the more astounding since the addition of gelatin had so far been expected to promote regeneration in elderly persons with morbid/degenerate cartilage structures.

However, the results found here indicate that the addition of collagen hydrolysate to the baby food produces effects which are totally independent of this.

In accordance with the invention, the use of collagen hydrolysate as supportive measure in the treatment and/or prevention of rickets in infants, particularly in combination with doses of vitamin D, is recommended.

The observed effects are so clearly evident that even small daily doses, for example, 0.5 g per day, can produce an effect.

On the other hand, owing to the nature of the collagen hydrolysate, even significantly higher doses than 1 g per day can virtually not result in overdosing. Accordingly, doses of up to 2.5 g of collagen hydrolysate based on the daily requirement of baby food can be readily recommended.

Furthermore, no undesired effects such as, for example, increased body growth, intolerance in the gastro-intestinal tract, etc., are observed.

In accordance with the invention, the collagen hydrolysate is used with a mean molecular weight Mw in the range of from 500 to 15,000, preferred from 1,000 to 6,000, further preferred from 1,500 to 5,000.

Collagen hydrolysates obtained in an enzymatic proteolytic manner are particularly preferred. In this type of collagen hydrolysate the proportion of bitter substances is minimal.

A further important criterion in selecting collagen hydrolysate for use as dietary supplement to baby food is cold-solubility in water, i.e., solubility to a clear solution at 23° C. In particular, this has advantages for use and administration together with the baby food.

### BRIEF DESCRIPTION OF THE SEVERAL VIEWS OF THE DRAWING(S)

FIG. 1 shows the results of a double-blind, randomized and placebo-controlled study for babies in the verum and placebo groups.

FIG. 2 shows a schematic structure a matured hip joint in sectional view.

FIG. 3 shows the results of hip sonography for a baby in which the femoral head core is fored.

FIG. 4 shows the results of hip sonography for a baby in which the femoral head core is not yet formed.

FIG. 5 is a graph showing the hip types combined according to normal and deviating findings for babies in the verum and placebo groups.

### DETAILED DESCRIPTION OF THE INVENTION

Description of the Results of the Study

42 babies out of originally 49 were finally included in a double-blind, randomized and placebo-controlled study. These underwent a routine check-up in the course of the preventive medical examinations in the 4^{th} to 6^{th} week of life and in the 14^{th} to 16^{th} week of life or at vaccination appointments in the doctor's surgery. Only healthy babies aged between 3 and 5 weeks were selected.

7 babies out of the original 49 were excluded: 4 on account of change of residence or non-compliance (2 in the verum group, 2 in the placebo group), 2 on account of administration problems (in the verum group), 1 on account of alleged intolerance (in the verum group). Hence n=21 babies remained in each of the verum and placebo groups.

There was no difference between the two groups as far as the basic demographic data of the mothers such as calendar age and gestational age, stimulants such as coffee, tea, alcohol and tobacco, and social and educational status were concerned. Nor were there any differences between the groups in respect of the basic data of the babies such as weight at birth, size, circumference of the head at birth, gender distribution, incidence of malformation, course of labour and type of delivery. Both groups showed good acceptance in handling and administering the test solutions.

In both study groups, the babies were fed in a similar way and, proportionally, breast feeding, mother's milk and hypoallergenic baby milk, mother's milk and follow-on milk as follow-up to baby starting milk, exclusively baby starting milk and hypoallergenic baby milk, were divided in a similar way between the groups in view of the various times at which the examinations were carried out.

It may be assumed that different nutritional concepts did not have any significant influence on the growth development of the babies in the present study.

The physical examinations of the infants in the preliminary examination, the examination 5 weeks, and the examination 9 weeks after administration of the test substance, regarding the internal condition of heart, lung, abdominal organs, genital organs, ear, nose and throat, skin, skeleton, skull, nervous system and sense organs, showed no differences within the groups at the various times at which the examinations were conducted and no differences between the groups.

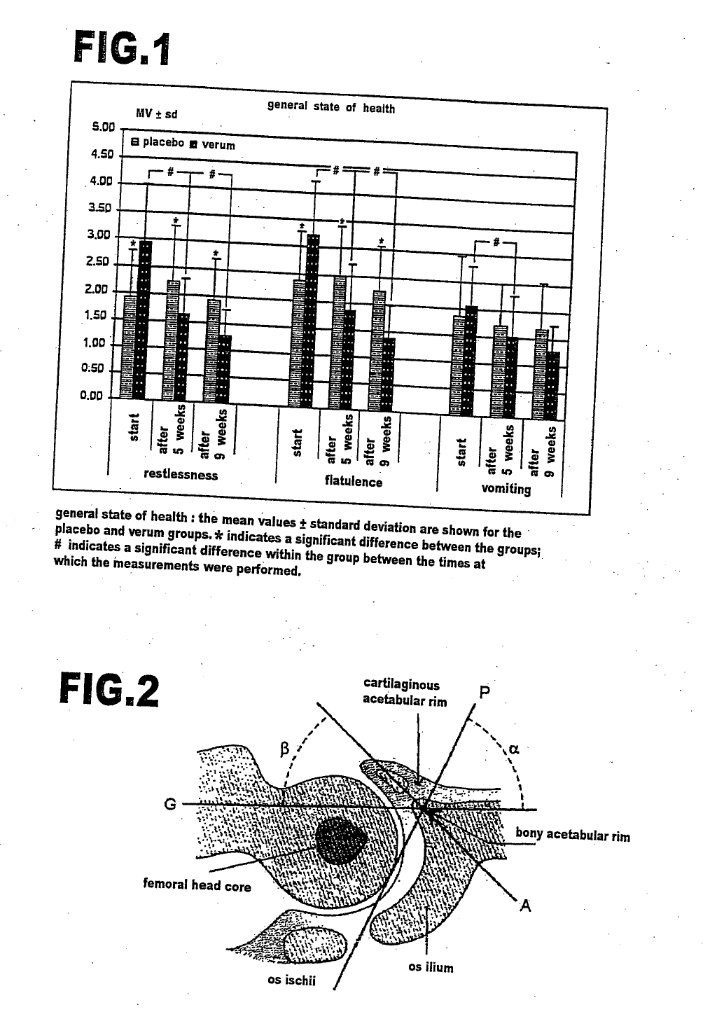

The findings for the two groups over the cited measurement times in respect of the parameters relevant to the general state of health such as restlessness, flatulence, vomiting and bowel movement, were as follows: regarding bowel movement a slight reduction in complaints was noted over the entire measurement time; there were no differences between the groups. As shown in FIG. 1, the two groups differ significantly in respect of the parameters restlessness and flatulence at the beginning of the study: in both respects, the verum group showed a significantly higher (worse) initial level. After start of administration of the test substance, a clearly different development occurred in the two groups: whereas the placebo group showed no change as regards restlessness, flatulence and vomiting over the measurement times, the values of the verum group improved to a highly significant extent. Similarly, comparison between the groups after 5 weeks and 9 weeks, respectively, of administration of the test substance was highly significant: the babies of the verum group were evaluated significantly better for the parameters restlessness and flatulence.

No difference in well-being in the subgroups formed with and without nicotine abuse and breast feeding in comparison with other forms of nutrition was found in terms of the parameters cited in connection with well-being.

To evaluate the general state of health of the babies, the parents were asked to assess the respective parameter on a scale of 1 to 5.

The following significance was allocated to the numbers: 1=none, 2=little, 3=average, 4=severe, 5=very severe.

Hip sonographies were carried out in accordance with the international criteria according to Graf (Graf, R. “Kursus der Hüftsonographie beim Säugling”, Fischer-Verlag, Stuttgart, 1995).

The hip sonography was carried out in a standardized manner in a positioning basin according to the guidelines of Graf[11,11]. A 7.5 MHz linear scan (Ultramark II+) was used as transducer; in each case, two images were frozen and then printed on a video printer on a scale of 2:1 and the findings interpreted. In analogy with the criteria according to Graf, alpha and beta angles and the following descriptive features were determined: bony formation, bony acetabular rim, roof of the acetabulum, cartilaginous roof, femoral head position and femoral head core. The hip type was thereby determined. The point in time of the sonographically determinable occurrence of ossification of the femoral head core was included as additional criterion for the maturing of the infant hip.

The results of the hip sonography did not show any significant differences between the groups either for the alpha and beta angles (see FIG. 2) right and left or for the qualities of the morphological description, bony formation, bony acetabular rim, formation of the roof of the acetabulum and cartilaginous roof. A development of the hip type right and left into hip type Ia was evident in both groups. In the classification according to hip types, there were not any significant differences, at any point in time, between the frequency of Ia, Ib and other hip types in the two study groups.

TABLE 1
hip types combined according to normal and
deviating findings for the two groups

| | | placebo | | verum | |
|---|---|---|---|---|---|
| | | Ia + Ib | | Ia + Ib | |
| | | normal | others | normal | others |
| | | findings | deviating | findings | deviating |
| hip type | start | 17 | 4 | 19 | 2 |
| right | after 5 weeks | 20 | 1 | 20 | 1 |
| | after 9 weeks | 21 | 0 | 20 | 1 |
| hip type | start | 18 | 3 | 18 | 3 |
| left | after 5 weeks | 20 | 1 | 21 | 0 |
| | after 9 weeks | 21 | 0 | 21 | 0 |

Differences did, however, occur between the groups with respect to both the number of proven femoral head cores and the point in time at which the femoral head cores formed. After 5 weeks of administration of the test substance, significantly more femoral head cores occurred in the verum group than in the placebo group. This effect also tends to remain after 9 weeks of administration of the test substance. Numerically, this effect emerges more clearly when left and right sides are subjected to a joint evaluation. The level of significance of this difference lies at 6%. Similarly, the growth of the femoral head cores is significantly greater in the verum group than in the placebo group.

After a further five weeks, the second physical examination including a hip sonography and the second investigation as to growth and general state of health were carried out at an appointment for vaccination. At the age of approximately 14 to 16 weeks, a physical examination and a hip sonography were then carried out on all patients, and a comprehensive anamnesis was recorded with respect to acceptance, side effects and development.

The schematic structure of a matured hip joint is contained in sectional representation in FIG. 2 with the definition of the above-mentioned alpha and beta angles.

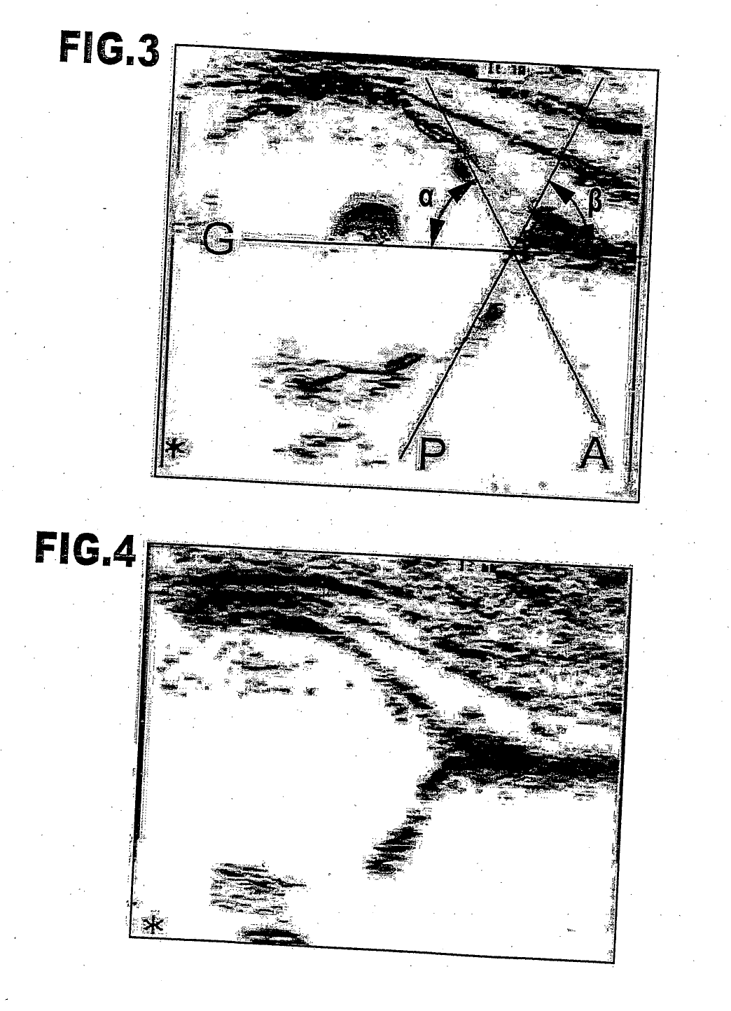

FIGS. 3 and 4 show the sonographic findings in

a) a baby in which the femoral head core is formed and

b) a baby in which the femoral head core is not yet formed.

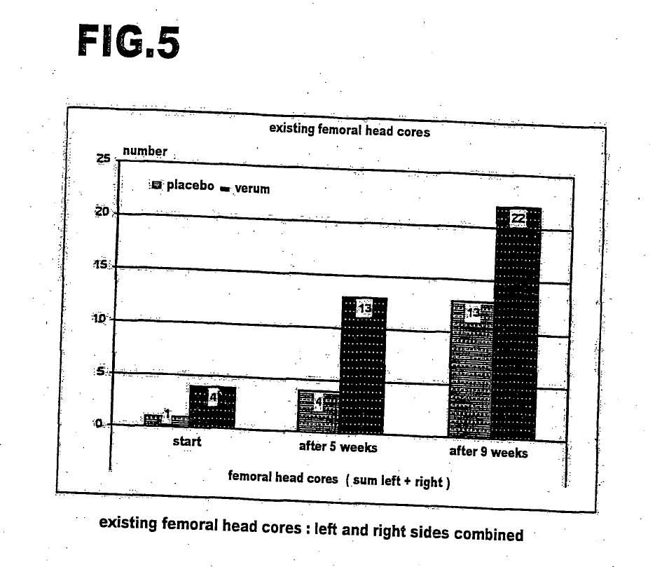

Finally, the data of Table 1 are represented graphically in FIG. 5.

The parents received as daily dietary supplement the numbered test substance in powder form, i.e., gelatin hydrolysate of the Gelitasol D type (Gelita Deutschland GmbH, Eberbach, Germany) or a placebo in the form of lactose monohydrate/Aerosil. The parents were asked to ask to add once daily for 10 weeks a 1 g measuring spoonful to the baby food. As form of administration, it was suggested that the powder be dissolved in water or milk and be directly orally dispensed by means of a pipette simultaneously with a dose of the vitamin D prophylactic (500 IU.(Vitamin D-Fluoretten)).

1. A dietary supplement to baby food comprising collagen hydrolysate.

2. The dietary supplement in accordance with claim 1, the collagen hydrolysate having a molecular weight Mw lying in the range of from 500 to 15,000.

3. The dietary supplement in accordance with claim 2, the collagen hydrolysate having a molecular weight Mw from 1,500 to 5,000.

4. The dietary supplement in accordance with claim 1, the collagen hydrolysate being soluble in water at 23° C. so as to form a clear solution.

5. The dietary supplement in accordance with claim 1, the collagen hydrolysate being produced in an enzymatic proteolytic process.

6. The method of claim 17 comprising feed the baby the dietary supplement to increase the general well-being of the baby.

7. The method of claim 17 comprising feeding the baby the dietary supplement to reduce restlessness of the baby.

8. The method of claim 17 comprising feeding the baby the dietary supplement to reduce flatulence of the baby.

9. The method of claim 17 comprising feeding the baby the dietary supplement to reduce the tendency of the baby to vomit.

10. The method of claim 17 comprising feeding the baby the dietary supplement to accelerate the mineralization of the skeleton of the baby.

11. The method of claim 17 comprising feeding the baby the dietary supplement to accelerate the maturing of the hips of the baby.

12. The method of claim 17 comprising feeding the baby the dietary supplement to accelerate the ossification of the femoral head of the baby.

13. The method of claim 17 comprising feeding the baby the dietary supplement as a supportive measure in the treatment and/or prevention of rickets of the baby.

14. The method of claim 17 comprising feeding the baby the collagen hydrolysate being added to the baby food.

15. The method of claim 14, wherein the amount of collagen hydrolysate added to the baby food is at least 0.5 g on the basis of the daily requirement of baby food.

16. The method of claim 15, wherein the amount of collagen hydrolysate added to the baby food is up to 2.5 g on the basis of the daily requirement of baby food.

17. A method of treating a baby comprising:
feeding a baby a dietary supplement comprising collagen hydrolysate.

18. The dietary supplement of claim 2, wherein the collagen hydrolysate has a molecular weight Mw in the range from 1,000 to 6,000.

19. The method of claim 13, including feed the baby doses of vitamin D.

20. The dietary supplement of claim 1, further comprising vitamin D.

21. The dietary supplement of claim 1, further comprising baby food.

22. The dietary supplement of claim 20, further comprising baby food.
